(19) United States
(12) Patent Application Publication
(10) Pub. No.: US 2010/0137754 A1
(43) Pub. Date: Jun. 3, 2010

(54) SHOCK WAVE LITHOTRIPTER SYSTEM AND A METHOD OF PERFORMING SHOCK WAVE CALCULUS FRAGMENTATION USING THE SAME

(76) Inventor: Yufeng Zhou, Seattle, WA (US)

Correspondence Address: Yufeng Zhou, 14041 15th Avenue NE, Apt. 310A, Seattle, WA, 98125, US

(21) Appl. No.: 12/522,754

(22) PCT Filed: Jan. 10, 2007
(86) PCT No.: PCT/CN07/00103

§ 371 Date: Jul. 10, 2009

(51) Int. Cl. A61B 17/225

(52) U.S. Cl. 601/4

(57) ABSTRACT

The present invention provides a shock wave lithotripter system and a method of performing shock wave calculus fragmentation using the same, the system is composed of current commercial lithotripter with protection device made of special acoustic material. The protection device could change the diffraction wave produced at the edge of the shock wave reflector, focusing lens, or shock wave generator, leading to the reduction of the energy of tensile wave in the focal region and the subsequent bubble cavitation effect in the renal tissue. Therefore, the propensity of shock wave induced tissue injury could be suppressed. Meanwhile, with the increase of the number of shock wave delivered, the effect of bubble cavitation effect be restored gradually to guarantee the success of stone fragmentation. The present invention can be used in treating all kinds of calculus diseases and in physical therapies. The present invention can be used in all commercial lithotripters, no matter of their methods of shock wave generation (electrohydraulic, electromagnetic, piezoelectric or combined method); the diverse method (blocking, attenuating, scattering, pressure inverting, etc) can be used to change the diffraction wave.

### RELATED APPLICATIONS

This application claims the benefit of Patent Cooperation Treaty Application Serial No. PCT/CN2007/000103, filed Jan. 10, 2007; the disclosure of which is incorporated herein by reference in its entirety.

### GOVERNMENT INTEREST

This invention was made with Government support under Grant No. 1 R41 DK072910-01 awarded by the National Institutes of Health. The Government has certain rights in the invention.

### TECHNICAL FIELD

The present invention belongs to medicinal field, relates to a shock wave lithotripter system and a method of performing shock wave calculus fragmentation using the same, and is primarily suitable for a variety of shock wave calculus fragmentation therapies.

### BACKGROUND OF THE INVENTION

Since its invention in the early of 1980s, extracorporeal shock wave lithotripsy (SWL) has been used routinely as a major modality for the fragmentation and comminution of kidney and ureteral stones in clinic. Despite its worldwide success in modern medical technology, both clinical and animal studies have also demonstrated that SWL is also accompanied by some forms of renal injury, such as hematuria, formation of diffuse hemorrhage and multiple hematomas within the renal parenchyma, perirenal fat, and subcapsular connective tissue, as well as kidney edema. Furthermore, the chronic consequence of SWL on renal function is still under investigation. Renal injury in SWL is primarily vascular lesions, characterized by extensive damage of the endothelial cells and the rupture of blood vessels, with capillary and small blood vessels much more susceptible to SWL injury than large vessels. In young adult patients, the vascular injury associated with SWL only affects about 2˜5% of their functional renal mass. Although most patients with normal renal function recover well following lithotripsy without significant clinical consequences, there are subgroups of patients who are at much higher risk for chronic SWL injury. These include patients with solitary kidneys, pre-existing hypertension, and, in particular, pediatric and elderly patients. Therefore, it is highly desirable to improve the safety of SWL treatment. Therefore, enhancing the safety of the lithotripsy is important and pressing, and is also one of the key points in the development of lithotripsy technology.

Basic investigation found that one primary mechanism of vascular injury in SWL is the mechanical dilation of the capillaries and small blood vessels caused by the large, rapid intraluminal expansion of cavitation bubbles. If such a large intraluminal bubble expansion is suppressed, for example by the inversion of the lithotripter shock waveform, vascular injury will be reduced. Unfortunately, inverted lithotripter shock waves do not break up kidney stones; and therefore cannot be successfully used for SWL. Clearly, there is a great need in SWL that can significantly suppress cavitation bubble expansion while maintaining effective stone communition efficiency.

In-situ pulse superposition method, imposing and almost compressive wave from the uncovered bottom of the orginal reflector on the tensile part of the lithotripter shock wave from a reflector insert, can suppress the bubble cavitation induced by the shock wave. The reflector insert is fitted on the commercial lithotripter and covered most of the inner surface of the lithotripter reflector. It has been proved both in vitro and in vivo experiments that in-situ pulse superposition method can suppress intraluminal bubble expansion with significant reduction of renal injury but no reduction of stone communition. However, this method needs re-design on each type of lithotripter for optimal geometrical parameters and the inter-pulse delay time. In addition, installation and removal is not very easy for different options of lithotripter. Therefore, an easy method for almost all lithotripters no matter their geometries and ways of generating shock waves are more suitable for clinical implementation and popularization.

The tensile component of the lithotripter shock wave is the major cause of the shock wave-induced bubble cavitation. From high-speed images, it is found that once the contact of bubble and vessel wall is established, the expansion of bubble would be limited and most of the kinetic energy would be absorbed by the vessel wall, leading to the dilation of the vessel. When the dilation exceeded the threshold of the vessel wall, vessel would be broken, which is the major mechanism of shock wave-induced tissue injury. In the free field, the maximum radium which bubble can reach (Rₘₐₓ) is far larger than the vessel size. So in the first order of approximation, the energy absorbed b the vessel is proportional to the Rₘₐₓ³. So an small amount suppression on Rₘₐₓ (30%) could lead to a significant reduction on the energy of vessel wall dilation (66%).

The propagation of shock wave in the acoustic field can be described simply as:

$$\frac{{p}_{2}}{{p}_{0}} = {H}_{c} ( z ) f ( {τ}_{c} ) + {H}_{e} ( z ) f ( {τ}_{e} ) + \frac{{C}_{0}}{a} {\int }_{{t}_{1}}^{{t}_{2}} {H}_{w} ( z , {t}^{′} ) f ( t - {t}^{′} ) d {t}^{′} \qquad ( 1 )$$

The waveform has three components (the right side of the equation 1): central wave (c), edge wave (e) and the wake (w). Although nonlinear equation can be used to describe the shock wave propagation more accurately, the simulated output still has these three components. In the lithotripter field, the central wave and the wake are close to each other. Initially, the diffraction wave generated at the edge of the lithotripter aperture (edge wave) is behind the central wave and the wake. While propagating towards the focal region of the lithotripter, the edge wave moves closer to the central wave and imposes on the wake as tensile wave. In comparison to the wake, the edge wave is the major component of the tensile wave and is the major mechanism of bubble caivtaiton. Theoretical studies show that bubble cavitaiton induced tissue injury can be reduced by changing the pressure waveform profile and order.

Except the tissue injury, the efficiency of calculus fragmentation is another criterion for evaluating the performance of lithotripter treatment. Calculus fragmentation is the results due to the stress wave propagating inside the calculus and the erosion effect on calculus surface because of bubble cavitation. In in vivo experiments, the contribution of these two effects has been studied. It is found that the stress wave can break calculus into pieces and plays a major role at the initial stage of treatment. However, when the size of fragments become small than 4˜8 mm, its effects will be limited. Although bubble cavitation can only spall small pieces from the calculus surface, it can change the structure of the calculus, making it weaker for the consequent shock waves. The bubble cavitation becomes more important at the later stage of lithotripsy treatment. Overall, these two mechanisms work synergistically rather than independently to ensure the success of lithotripsy. Therefore, when the erosion effect of bubble cavitation becomes dominant, bubble cavitation should be restored in order to comminute the calculus into small enough fragments which can be removed out of human body.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description, taken in connection with the drawings described in detail below.

FIG. 1 is a schematic drawing of a shock wave lithotripter system according to the present invention, showing a rotatable protection device retrofitted with a DORNIER HM-3 lithotrpter.

FIG. 2 is the top view of the shock wave lithotripter system, showing the rotatable protection device retrofitted with a DORNIER HM-3 lithotripter.

FIG. 3 is a graph showing representative pressure waveforms of the lithotripter shock waves generated by using (a) the original DORNIER HM-3 reflector; and (b) the DORNIER HM-3 reflector equipped with the protection device (full array); both at an output voltage of 20 kV, and for convenience, the offset between the two is set for 50 MPa.

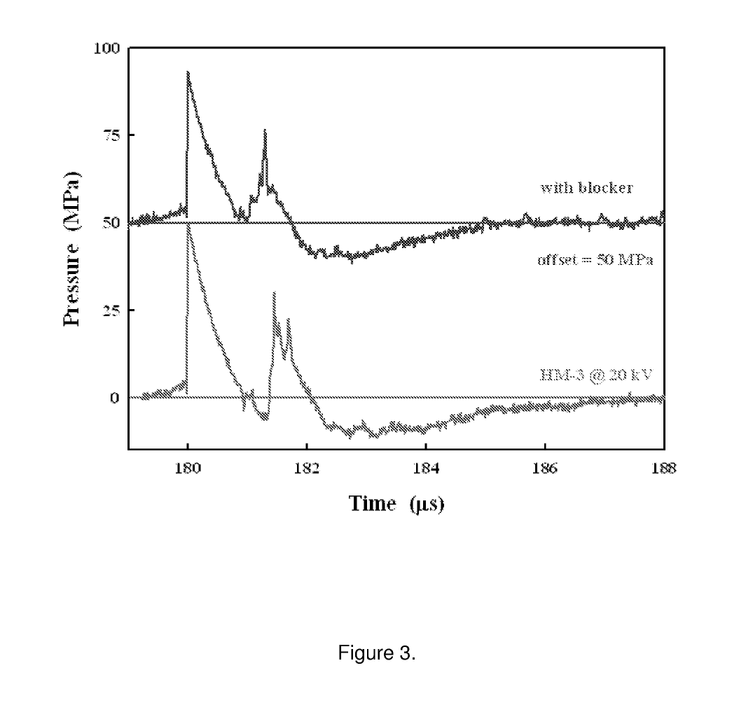

In the figures, all reference signs are described as follows:

1. Dornier HM-3 shock wave lithotripter;

2. The first focus of the ellipsoidal reflector (inner focus);

3. The ellipsoidal reflector of electrohydraulic shock wave lithotripter;

4. The generated shock wave inside the ellipsoidal reflector;

5. The shock wave arriving the edge of the reflector aperture;

6. The bracket of the protection device;

7. The rotatable or movable protection device;

8. The second focus of the ellipsoidal reflector (outer focus or the lithotripter focus);

9. The mechanical or electromechanical rotating or moving device, which can be adjusted automatically or manually.

### SUMMARY OF THE INVENTION

The present invention provides a shock wave lithotripter system and a method of performing shock wave calculus fragmentation using the same, the system is composed of current commercial lithotripter with protection device made of special acoustic material. The protection device could change the diffraction wave produced at the edge of the shock wave reflector, focusing lens, or shock wave generator, leading to the reduction of the energy of tensile wave in the focal region and the subsequent bubble cavitation effect in the renal tissue. Therefore, the propensity of shock wave induced tissue injury could be suppressed. Meanwhile, with the increase of the number of shock wave delivered, the effect of bubble cavitation should be restored gradually to guarantee the success of stone fragmentation. The present invention can be used in treating all kinds of calculus diseases and in physical therapies. The present invention can be used in all commercial lithotripters, no matter of their methods of shock wave generation (electrohydraulic, electromagnetic, piezoelectric or combined method); the diverse method (blocking, attenuating, scattering, pressure inverting, etc) can be used to change the diffraction wave.

### THE METHOD OF THE INVENTION

The key features of the current invention involve the specific modification on the diffraction wave from the edge of lithotripter, independent of how the shock waves are produced, either electrohydraulic, electromagnetic, or piezoelectric means, or combinations thereof, for generating shock waves. The application of the protection device on Dornier HM-3 lithotripter is used as an example to explain how the presented invention works.

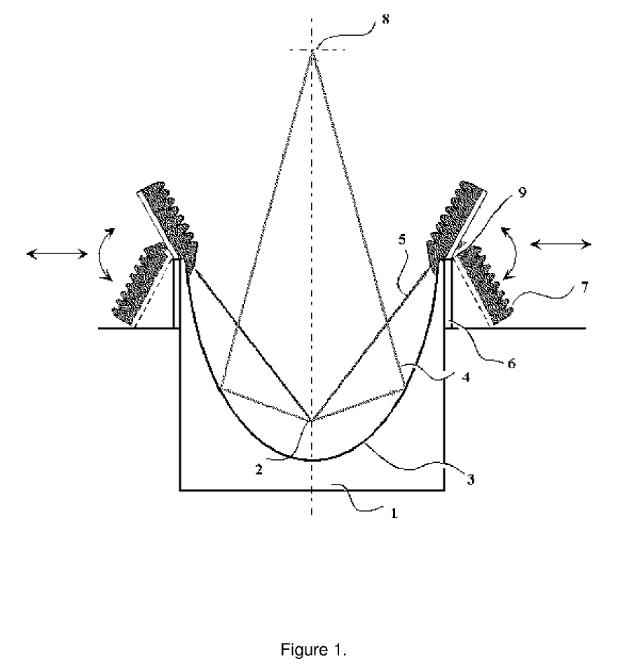

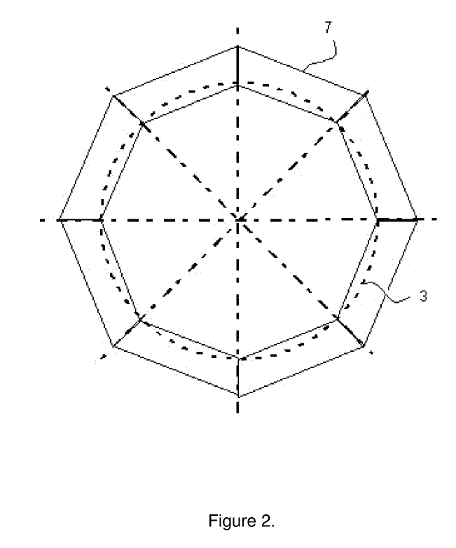

Referring to FIGS. 1 and 2, the DORNIER HM-3 lithotripter, which is one of the most widely used clinical lithotripters in the world, consists essentially of spark gap electrode and a truncated ellipsoidal reflector to focus the shock wave produced by the electrode discharge and the protection device 7 is installed on the bracket 6, which is fixed on the outer surface of ellipsoidal reflector 3 of lithotripter 1. The protection device 7 can also be directly connected with the lithotripter 1 without bracket 6, namely, the bracket 6 is not essential for the system. In the FIG. 2, the protection device 7 consists of eight protection modules, and in the event that there is provided with a bracket, each of which can rotate or move via the rotating or moving device 9 on the bracket 6. Special acoustic material is selectively attached on the front surface of protection device 7 to modify the diffraction wave. Before the start of lithotripsy treatment, lithotripter should have already installed with the protection device 7, which covers part of the outer coupling space and the special acoustic material thereon covers or does not cover inner surface of the reflector of the lithotripter 1. The shock waves generated at the first focus 2 of the ellipsoidal reflector 3 arrive on the inner surface of the reflector, and then are focused towards the second focus 8, where the calculus is aligned. The diffraction wave is generated at the edge of the reflector aperture and can be blocked, attenuated, scattered, or inverted by the special acoustic material on the front surface of the protection device 7. As a result, the tensile wave at the lithotripter focus 8 is suppressed. After a certain number of shock wave delivery, the calculus becomes fragments as shown in the fluoroscopic or ultrasound images. At this time, the effect of bubble cavitation needs to be restored, which is realized by increasing the angle or distance between the protection device 7 and the lithotripter 1 or both. When the size of calculus is smaller than 4˜8 mm, the protection device 7 has been far away from the lithotripter 1 or never covers the outer coupling space of lithotripter 1, that is, protection device 7 no longer changes the diffraction wave.

In in vitro experiment, the bubble collapse time at the lithotripter focus 8 can be reduced significantly with the protection device 7. The bubble collapse time of the original lithotripter is 322 μs, the values of using in-situ pulse interposition and protection device are only 268 and 160 μs, respectively. Therefore, the tissue injury can be reduced. In vessel rupture experiments, after 30 shocks the vessel phantom will be broken by using the original lithotripter. However, the vessel phantom is still intact after 200 shocks with the protection device. The diffraction wave at the lithotripter focus 8 can be up to 20 MPa and up to 200 μs in duration; and the protection device can reduce up to 90% of the diffraction wave.

Meanwhile the environment noise produced by the shock wave generator can be reduced significantly when applying the protection device. So the body movement caused by the pain or panic of stone patient can be reduced. It is known that when the calculus is away from the lithotripter focus, the treatment efficiency will become worse. Thus, when patient is calm, it's better not only for the treatment but also for recovery. In addition, the reduction of environment noise will also benefit clinical staffs for their working safety.

In the present invention, the lithotripsy treatment for reduced tissue injury and successful calculus fragmentation as well includes the following key steps: (1) generating shock waves; (2) shock waves are focused at the lithotripter focus 8; the diffraction wave produced at the edge of reflector aperture consists mostly of the tensile component of the shock wave; (3) restoring the erosion effect of bubble cavitation on the surface of calculus. The present invention can be applied not only in the treatments of all stone diseases in kidney, urinary tract, gallbladder, etc. in the body but also in physical therapies, such as all kinds of bone spur, myositis, desmitis, bone fracture, bone calcification or myocardial ischemia diseases.

The modification of the diffraction wave at the lithotripter edge is independent on the method of shock wave generation, so the present method can be used in all kinds of commercial lithotripters. The shock wave lithotripter can be electrohydraulic machine, piezoelectrical machine, electromagnetic machine, or combined type).

The protection device 7 is connected with the outer surface of lithotripter 1 and does not affect the operation of lithotripter 1 whether the protection device rotates or no rotates. In addition to this, the protection device 7 also does not need complex installation or removal. It will be appreciated that although the protection device 7 shown in the figures is composed of eight protection modules (up to 100 parts). The special acoustic material covers the inner surface of reflector 3, acoustic lens or piezoelectric shock wave generator for up to 50% of area or does not cover the same, and extends outwards outer coupling space for at least 1 mm. The special acoustic material is composing of foam, plastic, rubber, porous material, fiber polymer composite, which has at least 0.1 dB attenuation for the acoustic wave in the frequency range from 10 kHz to 100 MHz and thickness of at least 1 mm.

The protection device 7 can be rotated, such that the angle between it and the lithotripter axis varies from −90° (protection device lying horizontally on the surface of reflector aperture with special acoustic material facing the first focus of the reflector) to 180° (the protection device matching with the outer surface of lithotripter reflector 3 with special acoustic material facing outwards). In the initial stage of treatment, the angle is usually 0°˜90°. With the progress of the treatment, the angle between the protection device 7 and the lithotripter 1 axis can be gradually increased via mechanical or electromechanical device. So in the later stage of treatment, the angle may reach or exceed 90°, without modification effect on the diffraction wave from the edge of the reflector aperture. In addition, the protection device can also be moved away from the lithotripter in order to restore the bubble cavitaiton effects after the angle reaches or exceed 90°, and the distance between the protection device 7 and the lithotripter 1 varies up to 30 cm. After the completion of treatment, the protection device 7 will be restored to its initial status for the next operation.

The modification of diffraction wave at the edge of lithotripter aperture can be realized by using many methods, such as blocking, attenuating, scattering, waveform inverting.

The material of the bracket and the back-supporting for the protection device 7 can be, but not limited to, metal, wood, plastic rubber, ceramic, macromolecular polymer, fiber-polymer, porous material and their composite. The shape of cross-section of the protection device can be polygon, round, ellipse or irregular arcs, and its surface can be concave, convex, flat, or irregularly planar. As shown in FIG. 1, the special acoustic material on the front surface of the protection device is saw-tooth, which is one of the methods for attenuating acoustic waves, although it can also be flat or other shape of surfaces. The special acoustic material can be but not be limited to metal, wood, plastic, foam, rubber, ceramic, fiber-polymer, porous material or composite material, which can block the diffraction wave, or has at least 0.1 dB attenuation to the acoustic wave with frequency from 10 kHz to 100 MHz, or invert the waveform profile by using the air balloon or materials with acoustic impedance lower than water, or scatter the diffraction wave.

The present invention does not have strict requirements on the size, structure, composition of the protection device 7, and thus it is easy to be designed, manufactured, and operated and can achieve the aim of suppressing the shock wave injury to the tissue.

The system can be an individual system to complete the calculus fragmentation, and also can be a lithotripter system which is equipped with the protection device fixed to the outer surface of the ellipsoidal reflector or the acoustic focusing lens or the piezoelectric shock wave generator of the lithotripter.

While the present invention has been described with certain preferred embodiments, it is obvious that one skilled in the art can make various equivalents and modifications to the invention without departing from the spirit and scope of the invention, and all these equivalents and modifications should be considered as within the present invention.

What is claimed is:

1. A shock wave lithotripter system, the system comprising a shock wave lithotripter 1 and a protection device 7.

2. The shock wave lithotripsy system as claimed in claim 1, wherein the protection device 7 is connected directly or via a bracket 6 with the outer surface of an ellipsoidal reflector 3, an acoustic focusing lens, or a piezoelectric shock wave generator of lithotripter 1, which protection device 7 covers part of the outer acoustic coupling space of the shock wave lithotripter 1 and is capable of rotating or moving automatically or manually by using a mechanical or electromechanical device 9.

3. The shock wave lithotripter system as claimed in claim 1 wherein the protection device 7 comprises one or several protection modules, which are made of back-supporting material and selectively fronts special acoustic material to block, attenuate, scatter, invert diffraction wave.

4. The shock wave lithotripter system as claimed in claim 2, wherein the material of bracket 6 and the back-supporting of the protection device 7 comprises metal, wood, plastic, rubber, ceramic, macromolecular-polymer, fiber-polymer, porous material and their composite.

5. The shock wave lithotripter system as claimed in claim 3, wherein the top view of the protection device 7 is polygonal, round, ellipsoidal, or irregular arc, and the surface of the protection device 7 is concave, convex, flat or irregularly planar.

6. The shock wave lithotripter system as claimed in claim 3, wherein the acoustic attenuating material is foam, plastic, rubber, porous material, macromelecular-polymer, fiber-polymer, which has at least 0.1 dB attenuation to acoustic wave in the frequency from 10 kHz to 100 MHz.

7. The shock wave lithotripter system as claimed in claim 1 wherein the protection device 7 covers up to 50% of the inner surface of the reflector and extends outwards outer coupling space for at least 1 mm.

8. The shock wave lithotripter system as claimed in claim 1 wherein the protection device 7 can rotate with respect to the axis of lithotripter 1 in the angle range of −90°˜180°, or move from the outer surface of the lithotripter 1.

9. The shock wave lithotripter system as claimed in claim 1 wherein the shock wave lithotripter 1 is electrohydraulic, electromagnetic, combination of electrohydraulic and electromagnetic combination of electrohydraulic and piezoelectric, combination of electromagnetic and piezoelectric, or combination of electrohydraulic, electromagnetic and piezoelectric shock wave generation system.

10. A method of performing shock wave calculus fragmentation using the system as claimed in claim 1, the method comprising the steps of:
(a) Installing the protection device 7, such that the protection device 7 covers part of the outer acoustic coupling space of the lithotripter 1 and the special acoustic material covers part of the inner surface of the reflector or does not cover the same;
(b) Producing the shock wave by the lithotripter 1, the shock wave propagates and is focused at the target region, and the diffraction wave produced at the edge of the lithotripter 1 aperture is modified by the protection device 7;
(c) Gradually increasing the angle or the distance between the protection device 7 and the axis of the lithotripter 1 by rotating or moving the protection device 7 in order to restore the contribution of the diffraction wave to the bubble cavitation when the calculus becomes fragments as shown in the fluoroscopic or ultrasound imaging after a certain number of shock waves delivered;
(d) Making the protection device 7 has been far away from the lithotripter 1 or never covers the outer coupling space of the lithotripter 1 when the fragments become smaller than 2˜8 mm, and maintaining the protection device 7 in this status until the end of the treatment;
(e) Restoring the protection device 7 to its initial status for the next operation after the completion of shock wave treatment.

11. The method of performing shock wave calculus fragmentation as claimed in claim 10, wherein in the step (c), the protection device 7 can automatically or manually rotate, move, or first rotate then move away from the lithotripter 1 by using mechanical or electromechanical device 9, such that the angle between the protection device 7 and the axis of the lithotripter 1 varies between −90° and 180°, and the distance between the protection device 7 and the lithotripter 1 varies between 0 and 30 cm.

12. The method of performing shock wave calculus shock wave fragmentation as claimed in claim 10, wherein the protection device 7 can change the diffraction wave by means of blocking, attenuating, scattering, or wave inverting.

13. The method of performing shock wave calculus fragmentation as claimed in claim 10, wherein the method can be used in the treatment of kidney, urinary tract, gallbladder, or other calculus disease.

14. The method of performing shock wave calculus fragmentation as claimed in claim 10, wherein the method can be used in the physical therapies of bone spurs, myositis, desmitis, bone fracture, bone calcification, or myocardial ischemia disease.
